(19) United States
(12) Patent Application Publication
(10) Pub. No.: US 2025/0034103 A1
(43) Pub. Date: Jan. 30, 2025

(54) METHOD FOR ISOMERIZING METHYLTETRAHYDROPHTHALIC ANHYDRIDE

(71) Applicant: NAN YA PLASTICS CORPORATION, TAIPEI (TW)

(72) Inventors: TE-CHAO LIAO, TAIPEI (TW); JUNG-JEN CHUANG, TAIPEI (TW); CHUNG-YU CHEN, TAIPEI (TW); JUNG-TSU WU, TAIPEI (TW)

(21) Appl. No.: 18/474,204

(22) Filed: Sep. 25, 2023

(30) Foreign Application Priority Data
Jul. 28, 2023 (TW) 112128259

(51) Int. Cl. C07D 307/89; B01J 31/12

(52) U.S. Cl. CPC C07D 307/89; B01J 31/121; B01J 2231/52; B01J 2531/12

(57) ABSTRACT

A method for isomerizing methyltetrahydrophthalic anhydride includes the following processes. A liquid reactant is provided. Under a condition of injecting nitrogen gas, an alkaline catalyst is added into the liquid reactant for an isomerization reaction, so as to obtain an isomerization product. The liquid reactant includes 3-methyltetrahydrophthalic anhydride and 4-methyltetrahydrophthalic anhydride. Based on a total weight of the isomerization product being 100 wt %, an amount of a methyltetrahydrophthalic anhydride dimer is lower than 1.6 wt %.

### CROSS-REFERENCE TO RELATED PATENT APPLICATION

This application claims the benefit of priority to Taiwan Patent Application No. 112128259, filed on Jul. 28, 2023. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a method for isomerizing methyltetrahydrophthalic anhydride, and more particularly to a method for isomerizing methyltetrahydrophthalic anhydride in which a methyltetrahydrophthalic anhydride dimer is difficult to be generated.

### BACKGROUND OF THE DISCLOSURE

Methyltetrahydrophthalic anhydride is a hardener commonly used in an organic acid anhydride epoxy resin. Methyltetrahydrophthalic anhydride has good thermal resistance and thermal stability. Even under a high temperature environment, methyltetrahydrophthalic anhydride can still have good physical properties and electrical properties.

In general, methyltetrahydrophthalic anhydride can be polymerized from piperylene, isoprene, and acid anhydride. In a conventional method for manufacturing methyltetrahydrophthalic anhydride, weighed reactants are added into a reactor all at once for Diels-Alder reaction (also referred to as a conjugated diene addition reaction) so as to form methyltetrahydrophthalic anhydride. Subsequently, methyltetrahydrophthalic anhydride and an acidic catalyst, such as sulfuric acid or p-toluenesulfonic acid, are reacted for isomerization reaction, so as to enhance properties of methyltetrahydrophthalic anhydride.

However, the high temperature during the isomerization reaction easily causes a dimerization of methyltetrahydrophthalic anhydride to occur, which decreases an isomerization yield of the final product. Therefore, how to improve steps to prevent the dimerization and increase the isomerization yield has become one of the important issues to be addressed in the industry.

### SUMMARY OF THE DISCLOSURE

In response to the above-referenced technical inadequacies, the present disclosure provides a method for isomerizing methyltetrahydrophthalic anhydride.

In order to solve the above-mentioned problems, one of the technical aspects adopted by the present disclosure is to provide a method for isomerizing methyltetrahydrophthalic anhydride. The method for isomerizing methyltetrahydrophthalic anhydride includes steps as follows. A liquid reactant is provided. Under a condition that nitrogen gas is injected, an alkaline catalyst is added into the liquid reactant for an isomerization reaction, so as to obtain an isomerization product. The liquid reactant includes 3-methyltetrahydrophthalic anhydride and 4-methyltetrahydrophthalic anhydride. Based on a total weight of the isomerization product being 100 wt %, an amount of a methyltetrahydrophthalic anhydride dimer is lower than 1.6 wt %.

In one of the possible or preferred embodiments, the liquid reactant is carried out for the isomerization reaction at a temperature ranging from 140° C. to 160° C.

In one of the possible or preferred embodiments, the liquid reactant is carried out for the isomerization reaction under a condition that nitrogen gas is continuingly injected, and a volume flow rate of the nitrogen gas ranges from 0.4 L/hour to 0.8 L/hour.

In one of the possible or preferred embodiments, the nitrogen gas is injected into the liquid reactant before adding the alkaline catalyst into the liquid reactant for the isomerization reaction so as to remove oxygen gas and water vapor present in the liquid reactant.

In one of the possible or preferred embodiments, the nitrogen gas is injected into the liquid reactant in a form of bubbles so as to remove oxygen gas and water vapor present in the liquid reactant.

In one of the possible or preferred embodiments, the liquid reactant is carried out for the isomerization reaction in a reactor, and the nitrogen gas is injected into the reactor so as to remove oxygen gas and water vapor present in the reactor.

In one of the possible or preferred embodiments, a weight ratio of trans-3-methyltetrahydrophthalic anhydride to cis-3-methyltetrahydrophthalic anhydride in the isomerization product ranges from 4:1 to 5:1.

In one of the possible or preferred embodiments, based on a total weight of the liquid reactant being 100 phr, an amount of the alkaline catalyst ranges from 0.5 phr to 1.5 phr.

In one of the possible or preferred embodiments, a weight ratio of trans-3-methyltetrahydrophthalic anhydride to cis-3-methyltetrahydrophthalic anhydride in the liquid reactant ranges from 1:4 to 1:7.

In one of the possible or preferred embodiments, a weight ratio of 3-methyltetrahydrophthalic anhydride to 4-methyltetrahydrophthalic anhydride in the liquid reactant ranges from 7:3 to 3:7.

Therefore, in the method for isomerizing methyltetrahydrophthalic anhydride of the present disclosure provided by the present disclosure, by virtue of “under a condition of that nitrogen gas is injected, adding an alkaline catalyst into the liquid reactant for an isomerization reaction, so as to obtain an isomerization product,” the probability of the formation of the methyltetrahydrophthalic anhydride dimer can be reduced, thereby further enhancing the isomerization yield of methyltetrahydrophthalic anhydride.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a flowchart of a method for isomerizing methyltetrahydrophthalic anhydride of the present disclosure; and

FIG. 2 is a schematic view of a device used for isomerizing methyltetrahydrophthalic anhydride of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

In order to prevent the dimerization of methyltetrahydrophthalic anhydride and a low yield caused thereby, the isomerization reaction of the present disclosure is carried out under a condition that nitrogen gas is injected. Accordingly, the dimerization of methyltetrahydrophthalic anhydride can be prevented, and the isomerization yield of methyltetrahydrophthalic anhydride is improved.

Specifically, the nitrogen gas is injected into a liquid reactant containing 3-methyltetrahydrophthalic anhydride and 4-methyltetrahydrophthalic anhydride, so as to remove oxygen gas and water vapor present in the liquid reactant. The injection and exhaust of the nitrogen gas can take away oxygen gas and water vapor present in the system. In addition, compared to injecting the nitrogen gas into the system, injecting the nitrogen gas into the liquid reactant achieves a better effect of removing oxygen gas and water vapor, such that a probability of dimerization of methyltetrahydrophthalic anhydride can be decreased.

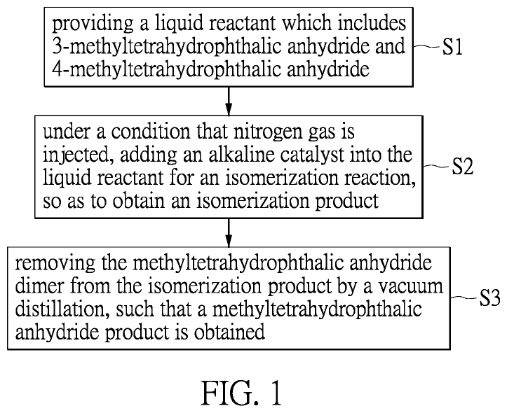

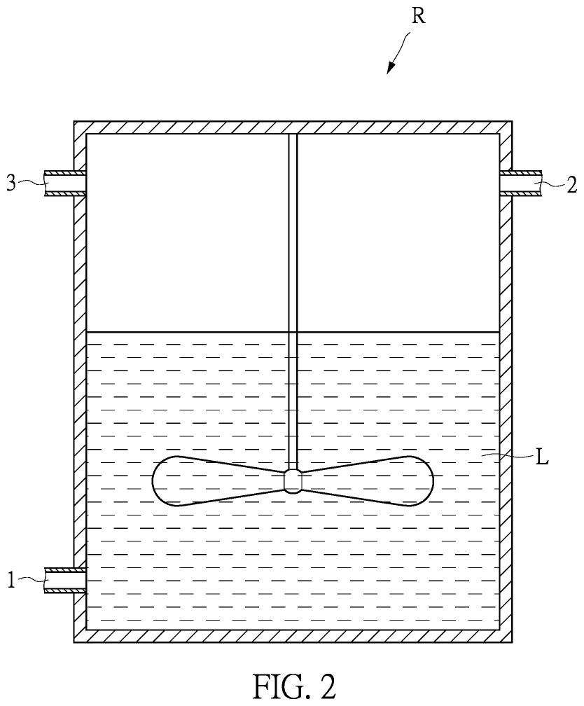

Referring to FIG. 1 and FIG. 2, FIG. 1 is a flowchart of a method for isomerizing methyltetrahydrophthalic anhydride of the present disclosure. FIG. 2 is a schematic view of a device used for isomerizing methyltetrahydrophthalic anhydride of the present disclosure. The device shown in FIG. 2 can be used to implement the method for isomerizing methyltetrahydrophthalic anhydride of the present disclosure, but is not used to limit steps in the method of the present disclosure.

In step S1, the liquid reactant is provided. The liquid reactant contains 3-methyltetrahydrophthalic anhydride and 4-methyltetrahydrophthalic anhydride.

Due to a difference in three-dimensional structures, 3-methyltetrahydrophthalic anhydride has optical isomers. Specifically, 3-methyltetrahydrophthalic anhydride includes trans-3-methyltetrahydrophthalic anhydride and cis-3-methyltetrahydrophthalic anhydride. In the liquid reactant, an amount of trans-3-methyltetrahydrophthalic anhydride is higher than an amount of cis-3-methyltetrahydrophthalic anhydride. For example, a weight ratio of trans-3-methyltetrahydrophthalic anhydride to cis-3-methyltetrahydrophthalic anhydride can be 1:4, 1:4.5, 1:5, 1:5.5, 1:6, 1:6.5, or 1:7. However, the present disclosure is not limited thereto.

More specifically, 3-methyltetrahydrophthalic anhydride in the liquid reactant can be synthesized from maleic anhydride and piperylene by an addition reaction. 4-methyltetrahydrophthalic anhydride in the liquid reactant can be synthesized from maleic anhydride and isoprene by an addition reaction.

In an exemplary embodiment, maleic anhydride and piperylene are added into a reactor, and reacted at a temperature ranging from 80° C. to 120° C. for a first addition reaction. In the first addition reaction, 3-methyltetrahydrophthalic anhydride is synthesized by maleic anhydride and piperylene. Subsequently, isoprene is added into the reactor, and reacted at a temperature ranging from 80° C. to 120° C. for a second addition reaction. In the second addition reaction, 4-methyltetrahydrophthalic anhydride is synthesized by maleic anhydride and isoprene.

In order to adjust the amounts of 3-methyltetrahydrophthalic anhydride and 4-methyltetrahydrophthalic anhydride, the first addition reaction is stopped when a conversion rate of maleic anhydride is higher than 25%. In other words, an expected generation amount of 3-methyltetrahydrophthalic anhydride of the present disclosure is higher than 25 wt %. Specifically, the first addition reaction is stopped when the conversion rate of maleic anhydride is 30%, 35%, 40%, 45%, 50%, 55%, 60%, 65%, 70%, 75%, or 80%. However, the present disclosure is not limited thereto.

A melting point of 3-methyltetrahydrophthalic anhydride is lower than a melting point of 4-methyltetrahydrophthalic anhydride. When the amount of 3-methyltetrahydrophthalic anhydride in the liquid reactant is low, the liquid reactant has a high viscosity. When the amount of 3-methyltetrahydrophthalic anhydride in the liquid reactant is high, the liquid reactant has a low viscosity. Therefore, the viscosity of the liquid reactant can be adjusted by changing the weight ratio of 3-methyltetrahydrophthalic anhydride to 4-methyltetrahydrophthalic anhydride, so as to facilitate subsequent steps, such as injecting nitrogen gas. For example, the viscosity of the liquid reactant can range from 50 cps to 100 cps. However, the present disclosure is not limited thereto.

In step S2, under a condition of injecting nitrogen gas, an alkaline catalyst is added into the liquid reactant for an isomerization reaction, so as to obtain an isomerization product.

In an exemplary embodiment, the isomerization reaction is carried out at a temperature ranging from 140° C. to 160° C. Before the isomerization reaction, the amount of trans-3-methyltetrahydrophthalic anhydride is lower than the amount of cis-3-methyltetrahydrophthalic anhydride. After the isomerization reaction, the amount of trans-3-methyltetrahydrophthalic anhydride is higher than the amount of cis-3-methyltetrahydrophthalic anhydride.

However, in the presence of oxygen gas and water vapor, oxygen gas and water vapor induce methyltetrahydrophthalic anhydride to open a double bond for dimerization. Under a high temperature environment or in the presence of initiators, methyltetrahydrophthalic anhydride that has an open double bond tends to react with another methyltetrahydrophthalic anhydride that has an open double bond to form a methyltetrahydrophthalic anhydride dimer.

In the present disclosure, the initiators are excluded from the method. However, the isomerization reaction is carried out at a high temperature (140° C. to 160° C.), which is appropriate for a dimerization of methyltetrahydrophthalic anhydride. In other words, the isomerization reaction can be accompanied by the dimerization of methyltetrahydrophthalic anhydride.

In order to prevent the formation of the methyltetrahydrophthalic anhydride dimer, nitrogen gas is injected into the system to remove oxygen gas and water vapor present in the system. Oxygen gas and water vapor present in the system may come from the addition of reactants or may be originally present in the device; hence, the presence of oxygen gas and water vapor are difficult to be avoided.

Referring to FIG. 2, a liquid reactant L is placed in a reactor R. Nitrogen gas is injected into the reactor R via an inlet pipe 1 which is located at a bottom of the reactor R. Nitrogen gas can be injected into the reactor R in a form of bubbles and replace the oxygen gas and water vapor originally present in the liquid reactant L. Subsequently, the nitrogen gas is exhausted via an outlet pipe 2 which is located at a top of the reactor R, and the oxygen gas and water vapor are exhausted together with the nitrogen gas.

In another embodiment, the nitrogen gas also can be injected into the reactor R via another inlet pipe 3 which is located at the top of the reactor R and exhausted from the reactor R via the outlet pipe 2. Therefore, the oxygen gas and water vapor are similarly exhausted together with the nitrogen gas. Moreover, the injection of the nitrogen gas causes a pressure difference between the inlet pipe 3 and the outlet pipe 2 of the reactor R. Therefore, the oxygen and water vapor in the liquid reactant L will be exhausted together with the nitrogen gas.

The above two ways for injecting nitrogen can both achieve the effect of removing oxygen and water vapor in the system. However, the present disclosure is not limited thereto.

Accordingly, oxygen and water vapor which may induce methyltetrahydrophthalic anhydride to open the double bond can be removed. Even when the isomerization reaction is carried out at 140° C. to 160° C., the formation of the methyltetrahydrophthalic anhydride dimer can be prevented.

Therefore, in order to prevent the formation of the methyltetrahydrophthalic anhydride dimer, nitrogen gas is continuously injected into the reactor R before a preheating for the isomerization reaction and throughout the entirety of the isomerization reaction, and the injection of the nitrogen gas is stopped after the isomerization reaction and the cooling of the reactant to room temperature.

Throughout the entirety of the isomerization reaction, a volume flow rate of the nitrogen gas can be between 0.4 L/hour and 0.8 L/hour, and preferably is 0.5 L/hour, 0.6 L/hour, or 0.7 L/hour. If the volume flow rate of the nitrogen gas is too low, an inhibition against the methyltetrahydrophthalic anhydride dimer is poor. If the volume flow rate of the nitrogen gas is too high, it is not economically beneficial for the overall process.

In an exemplary embodiment, the amount of the methyltetrahydrophthalic anhydride dimer can be measured by a gel permeation chromatography (DPC) (brand: SHIMADZU©, model: NEXERA LC40D GPC).

Based on a total weight of the isomerization product being 100 wt %, an amount of the methyltetrahydrophthalic anhydride dimer in the isomerization product is lower than 1.6 wt %. Specifically, the amount of the methyltetrahydrophthalic anhydride dimer can be 1.5 wt %, 1.4 wt %, 1.3 wt %, 1.2 wt %, 1.0 wt %, 0.8 wt %, 0.7 wt %, or 0.6 wt %.

The alkaline catalyst is used for the isomerization reaction in the present disclosure. The addition of the alkaline catalyst can decrease the viscosity of the hardener (isomerization product). Therefore, the hardener having a low viscosity can have a good processability and a wide range of applications. Specifically, the viscosity of the isomerization product can range from 30 cps to 40 cps.

For example, the alkaline catalyst can be an organic alkaline catalyst or an inorganic alkaline catalyst. The organic alkaline catalyst can be an alcoholamine compound, and more specifically a compound selected from the group consisting of ethanolamine, diethanolamine, and triethanolamine. The inorganic alkaline catalyst can be selected from the group consisting of potassium carbonate, sodium carbonate, sodium hydroxide, and potassium hydroxide.

After the oxygen gas and water vapor are removed, methyltetrahydrophthalic anhydride can be prevented from opening double bonds to form the methyltetrahydrophthalic anhydride dimer during the process of the isomerization reaction. Therefore, an amount of the alkaline catalyst can be increased. In an exemplary embodiment, based on a total weight of the liquid reactant being 100 phr, the amount of the alkaline catalyst can range from 0.5 phr to 1.5 phr.

After the isomerization reaction, the amount of trans-3-methyltetrahydrophthalic anhydride is higher than the amount of cis-3-methyltetrahydrophthalic anhydride in the isomerization product. A weight ratio of trans-3-methyltetrahydrophthalic anhydride to cis-3-methyltetrahydrophthalic anhydride can be measured by a gas chromatography (GC) (brand: AGILENT©, model: 8860 GC).

In an exemplary embodiment, the weight ratio of trans-3-methyltetrahydrophthalic anhydride to cis-3-methyltetrahydrophthalic anhydride can range from 3:1 to 6:1. Preferably, the weight ratio of trans-3-methyltetrahydrophthalic anhydride to cis-3-methyltetrahydrophthalic anhydride can range from 4:1 to 5:1. Specifically, the weight ratio of trans-3-methyltetrahydrophthalic anhydride to cis-3-methyltetrahydrophthalic anhydride can be 3.5:1, 4.2:1, 4.5:1, 4.7:1, 5.0:1, or 5.5:1.

In step S3, the isomerization product is carried out by a vacuum distillation to remove the methyltetrahydrophthalic anhydride dimer, such that a methyltetrahydrophthalic anhydride product is obtained.

Because only a small amount of the methyltetrahydrophthalic anhydride dimer is generated in the isomerization product, it takes a shorter period of time to purify the methyltetrahydrophthalic anhydride product during the vacuum distillation to achieve an expected purity. In addition, in a situation that the same amount of material is used, the method for isomerizing methyltetrahydrophthalic anhydride of the present disclosure can reach a high isomerization yield.

In order to illustrate the method for isomerizing methyltetrahydrophthalic anhydride of the present disclosure, Examples 1 to 7 and Comparative Examples 1 and 2 are prepared. The reaction parameters used in Examples 1 to 5 and properties that are measured are listed in Table 1. The reaction parameters used in Examples 6 and 7 and Comparative Examples 1 and 2 and properties that are measured are listed in Table 2.

### Example 1

10000 kg of a liquid reactant is added into a reactor. The liquid reactant includes 7500 kg of 3-methyltetrahydrophthalic anhydride and 2500 kg of 4-methyltetrahydrophthalic anhydride. A weight ratio of trans-3-methyltetrahydrophthalic anhydride to cis-3-methyltetrahydrophthalic anhydride contained in the liquid reactant is 1:6. In Table 1 and Table 2, the weight ratio of trans-3-methyltetrahydrophthalic anhydride to cis-3-methyltetrahydrophthalic anhydride contained in the liquid reactant is abbreviated as “trans/cis ratio of MTHPA” for illustration.

Nitrogen gas is injected into a bottom part of the reactor, and the nitrogen gas is directly injected into the liquid reactant in a form of bubbles, so as to remove oxygen gas and water vapor present in the liquid reactant. Under a condition of continuously injecting nitrogen gas, the reactor is heated to reach a temperature of 140° C., and then 150 kg of sodium hydride (alkaline catalyst) is added into the reactor for an isomerization reaction. In other words, based on a total weight of the liquid reactant being 100 phr, an additional amount of the alkaline catalyst is 1.5 phr, which is abbreviated as “a relative amount of the alkaline catalyst (based on the liquid reactant) being 1.5/100” for illustration.

During the process of the isomerization reaction, when the weight ratio of trans-3-methyltetrahydrophthalic anhydride to cis-3-methyltetrahydrophthalic anhydride measured by a GC is higher than 4:1, the isomerization reaction is stopped, and an isomerization product is obtained. Duration for the process of the isomerization reaction in Example 1 is 24 hours.

The isomerization product is measured by a GPC. Based on a total weight of the isomerization product being 100 wt %, an amount of the methyltetrahydrophthalic anhydride dimer is 1.1 wt %. After being measured by a GPC, the weight ratio of trans-3-methyltetrahydrophthalic anhydride to cis-3-methyltetrahydrophthalic anhydride contained in the isomerization product is 4.21:1.

### Example 2

10000 kg of a liquid reactant is added into a reactor. The liquid reactant includes 7500 kg of 3-methyltetrahydrophthalic anhydride and 2500 kg of 4-methyltetrahydrophthalic anhydride. A weight ratio of trans-3-methyltetrahydrophthalic anhydride to cis-3-methyltetrahydrophthalic anhydride contained in the liquid reactant is 1:6.

Nitrogen gas is injected into a bottom part of the reactor, and the nitrogen gas is directly injected into the liquid reactant in a form of bubbles, so as to remove oxygen gas and water vapor present in the liquid reactant. Under a condition of continuously injecting nitrogen gas, the reactor is heated to reach a temperature of 150°, and then 150 kg of sodium hydride (alkaline catalyst) is added into the reactor for an isomerization reaction.

During the process of the isomerization reaction, when the weight ratio of trans-3-methyltetrahydrophthalic anhydride to cis-3-methyltetrahydrophthalic anhydride measured by a GC is higher than 4:1, the isomerization reaction is stopped, and an isomerization product is obtained. Duration for the process of the isomerization reaction in Example 2 is 6 hours.

The isomerization product is measured by a GPC. Based on a total weight of the isomerization product being 100 wt %, an amount of the methyltetrahydrophthalic anhydride dimer is 0.53 wt %. After being measured by a GPC, the weight ratio of trans-3-methyltetrahydrophthalic anhydride to cis-3-methyltetrahydrophthalic anhydride contained in the isomerization product is 4.17:1.

### Example 3

10000 kg of a liquid reactant is added into a reactor. The liquid reactant includes 7500 kg of 3-methyltetrahydrophthalic anhydride and 2500 kg of 4-methyltetrahydrophthalic anhydride. A weight ratio of trans-3-methyltetrahydrophthalic anhydride to cis-3-methyltetrahydrophthalic anhydride contained in the liquid reactant is 1:6.

Nitrogen gas is injected into a bottom part of the reactor, and the nitrogen gas is directly injected into the liquid reactant in a form of bubbles, so as to remove oxygen gas and water vapor present in the liquid reactant. Under a condition of continuously injecting nitrogen gas, the reactor is heated to reach a temperature of 150°, and then 50 kg of sodium hydride (alkaline catalyst) is added into the reactor for an isomerization reaction.

During the process of the isomerization reaction, when the weight ratio of trans-3-methyltetrahydrophthalic anhydride to cis-3-methyltetrahydrophthalic anhydride measured by a GC is higher than 4:1, the isomerization reaction is stopped, and an isomerization product is obtained. Duration for the process of the isomerization reaction in Example 3 is 36.5 hours.

The isomerization product is measured by a GPC. Based on a total weight of the isomerization product being 100 wt %, an amount of the methyltetrahydrophthalic anhydride dimer is 0.79 wt %. After being measured by a GPC, the weight ratio of trans-3-methyltetrahydrophthalic anhydride to cis-3-methyltetrahydrophthalic anhydride contained in the isomerization product is 4.23:1.

### Example 4

10000 kg of a liquid reactant is added into a reactor. The liquid reactant includes 7500 kg of 3-methyltetrahydrophthalic anhydride and 2500 kg of 4-methyltetrahydrophthalic anhydride. A weight ratio of trans-3-methyltetrahydrophthalic anhydride to cis-3-methyltetrahydrophthalic anhydride contained in the liquid reactant is 1:6.

Nitrogen gas is injected into a bottom part of the reactor, and the nitrogen gas is directly injected into the liquid reactant in a form of bubbles, so as to remove oxygen gas and water vapor present in the liquid reactant. Under a condition of continuously injecting nitrogen gas, the reactor is heated to reach a temperature of 140°, and then 100 kg of sodium hydride (alkaline catalyst) is added into the reactor for an isomerization reaction.

During the process of the isomerization reaction, when the weight ratio of trans-3-methyltetrahydrophthalic anhydride to cis-3-methyltetrahydrophthalic anhydride measured by a GC is higher than 4:1, the isomerization reaction is stopped, and an isomerization product is obtained. Duration for the process of the isomerization reaction in Example 4 is 26 hours.

The isomerization product is measured by a GPC. Based on a total weight of the isomerization product being 100 wt %, an amount of the methyltetrahydrophthalic anhydride dimer is 1.24 wt %. After being measured by a GPC, the weight ratio of trans-3-methyltetrahydrophthalic anhydride to cis-3-methyltetrahydrophthalic anhydride contained in the isomerization product is 4.19:1.

### Example 5

10000 kg of a liquid reactant is added into a reactor. The liquid reactant includes 7500 kg of 3-methyltetrahydrophthalic anhydride and 2500 kg of 4-methyltetrahydrophthalic anhydride. A weight ratio of trans-3-methyltetrahydrophthalic anhydride to cis-3-methyltetrahydrophthalic anhydride contained in the liquid reactant is 1:6.

Nitrogen gas is injected into a bottom part of the reactor, and the nitrogen gas is directly injected into the liquid reactant in a form of bubbles, so as to remove oxygen gas and water vapor present in the liquid reactant. Under a condition of continuously injecting nitrogen gas, the reactor is heated to reach a temperature of 140°, and then 50 kg of sodium hydride (alkaline catalyst) is added into the reactor for an isomerization reaction.

During the process of the isomerization reaction, when the weight ratio of trans-3-methyltetrahydrophthalic anhydride to cis-3-methyltetrahydrophthalic anhydride measured by a GC is higher than 4:1, the isomerization reaction is stopped, and an isomerization product is obtained. Duration for the process of the isomerization reaction in Example 5 is 72 hours.

The isomerization product is measured by a GPC. Based on a total weight of the isomerization product being 100 wt %, an amount of the methyltetrahydrophthalic anhydride dimer is 1.31 wt %. After being measured by a GPC, the weight ratio of trans-3-methyltetrahydrophthalic anhydride to cis-3-methyltetrahydrophthalic anhydride contained in the isomerization product is 4.11:1.

### Example 6

10000 kg of a liquid reactant is added into a reactor. The liquid reactant includes 7500 kg of 3-methyltetrahydrophthalic anhydride and 2500 kg of 4-methyltetrahydrophthalic anhydride. A weight ratio of trans-3-methyltetrahydrophthalic anhydride to cis-3-methyltetrahydrophthalic anhydride contained in the liquid reactant is 1:6.

Nitrogen gas is injected into a bottom part of the reactor, and the nitrogen gas is directly injected into the liquid reactant in a form of bubbles, so as to remove oxygen gas and water vapor present in the liquid reactant. Under a condition of continuously injecting nitrogen gas, the reactor is heated to reach a temperature of 160°, and then 150 kg of sodium hydride (alkaline catalyst) is added into the reactor for an isomerization reaction.

During the process of the isomerization reaction, when the weight ratio of trans-3-methyltetrahydrophthalic anhydride to cis-3-methyltetrahydrophthalic anhydride measured by a GC is higher than 4:1, the isomerization reaction is stopped, and an isomerization product is obtained. Duration for the process of the isomerization reaction in Example 6 is 3.5 hours.

The isomerization product is measured by a GPC. Based on a total weight of the isomerization product being 100 wt %, an amount of the methyltetrahydrophthalic anhydride dimer is 1.55 wt %. After being measured by a GPC, the weight ratio of trans-3-methyltetrahydrophthalic anhydride to cis-3-methyltetrahydrophthalic anhydride contained in the isomerization product is 4.22:1.

### Example 7

10000 kg of a liquid reactant is added into a reactor. The liquid reactant includes 7500 kg of 3-methyltetrahydrophthalic anhydride and 2500 kg of 4-methyltetrahydrophthalic anhydride. A weight ratio of trans-3-methyltetrahydrophthalic anhydride to cis-3-methyltetrahydrophthalic anhydride contained in the liquid reactant is 1:6.

Nitrogen gas is injected into one end of a top part of the reactor, and the nitrogen gas is exhausted from another end of the top part of the reactor, so as to remove oxygen gas and water vapor present in the reactor. Under a condition of continuously injecting nitrogen gas, the reactor is heated to reach a temperature of 140°, and then 150 kg of sodium hydride (alkaline catalyst) is added into the reactor for an isomerization reaction.

During the process of the isomerization reaction, when the weight ratio of trans-3-methyltetrahydrophthalic anhydride to cis-3-methyltetrahydrophthalic anhydride measured by a GC is higher than 4:1, the isomerization reaction is stopped, and an isomerization product is obtained. Duration for the process of the isomerization reaction in Example 7 is 24 hours.

The isomerization product is measured by a GPC. Based on a total weight of the isomerization product being 100 wt %, an amount of the methyltetrahydrophthalic anhydride dimer is 1.52 wt %. After being measured by a GPC, the weight ratio of trans-3-methyltetrahydrophthalic anhydride to cis-3-methyltetrahydrophthalic anhydride contained in the isomerization product is 4.21:1.

### Comparative Example 1

10000 kg of a liquid reactant is added into a reactor. The liquid reactant includes 7500 kg of 3-methyltetrahydrophthalic anhydride and 2500 kg of 4-methyltetrahydrophthalic anhydride. A weight ratio of trans-3-methyltetrahydrophthalic anhydride to cis-3-methyltetrahydrophthalic anhydride contained in the liquid reactant is 1:6.

Under a condition of not injecting nitrogen gas, the reactor is heated to reach a temperature of 140°, and then 150 kg of sodium hydride (alkaline catalyst) is added into the reactor for an isomerization reaction.

During the process of the isomerization reaction, when the weight ratio of trans-3-methyltetrahydrophthalic anhydride to cis-3-methyltetrahydrophthalic anhydride measured by a GC is higher than 4:1, the isomerization reaction is stopped, and an isomerization product is obtained. Duration for the process of the isomerization reaction in Comparative Example 1 is 24 hours.

The isomerization product is measured by a GPC. Based on a total weight of the isomerization product being 100 wt %, an amount of the methyltetrahydrophthalic anhydride dimer is 4 wt %. After being measured by a GPC, the weight ratio of trans-3-methyltetrahydrophthalic anhydride to cis-3-methyltetrahydrophthalic anhydride contained in the isomerization product is 4.24:1.

### Comparative Example 2

10000 kg of a liquid reactant is added into a reactor. The liquid reactant includes 7500 kg of 3-methyltetrahydrophthalic anhydride and 2500 kg of 4-methyltetrahydrophthalic anhydride. A weight ratio of trans-3-methyltetrahydrophthalic anhydride to cis-3-methyltetrahydrophthalic anhydride contained in the liquid reactant is 1:6.

Under a condition of not injecting nitrogen gas, the reactor is heated to reach a temperature of 140°, and then 100 kg of sodium hydride (alkaline catalyst) is added into the reactor for an isomerization reaction.

During the process of the isomerization reaction, when the weight ratio of trans-3-methyltetrahydrophthalic anhydride to cis-3-methyltetrahydrophthalic anhydride measured by a GC is higher than 4:1, the isomerization reaction is stopped, and an isomerization product is obtained. Duration for the process of the isomerization reaction in Comparative Example 2 is 26 hours.

The isomerization product is measured by a GPC. Based on a total weight of the isomerization product being 100 wt %, an amount of the methyltetrahydrophthalic anhydride dimer is 3.72 wt %. After being measured by a GPC, the weight ratio of trans-3-methyltetrahydrophthalic anhydride to cis-3-methyltetrahydrophthalic anhydride contained in the isomerization product is 4.19:1.

TABLE 1

| | | Example | | | | |
|---|---|---|---|---|---|---|
| | | 1 | 2 | 3 | 4 | 5 |
| Liquid | Trans/cis | 1:6 | 1:6 | 1:6 | 1:6 | 1:6 |
| reactant | ratio | | | | | |
| | of MTHPA | | | | | |
| Process | Injection of | Yes | Yes | Yes | Yes | Yes |
| parameter | nitrogen gas | | | | | |
| | Temperature of | 140° C. | 150° C. | 150° C. | 140° C. | 140° C. |
| | isomerization reaction | | | | | |
| | Relative amount of | 1.5/100 | 1.5/100 | 0.5/100 | 1.0/100 | 0.5/100 |
| | the alkaline catalyst | | | | | |
| | Duration of | 24 | 6 | 36.5 | 26 | 72 |
| | isomerization reaction | hours | hours | hours | hours | hours |
| Isomerization | Trans/cis ratio | 4.21:1 | 4.17:1 | 4.23:1 | 4.19:1 | 4.11:1 |
| product | of MTHPA | | | | | |
| | Amount of | 1.1 | 0.53 | 0.79 | 1.24 | 1.31 |
| | methyltetrahydro- | wt % | wt % | wt % | wt % | wt % |
| | phthalic anhydride | | | | | |
| | dimer | | | | | |

TABLE 2

| | | Example | | | | Comparative Example | | | |
|---|---|---|---|---|---|---|---|---|---|
| | | 6 | | 7 | | 1 | | 2 | |
| Liquid | Trans/cis | 1:6 | | 1:6 | | 1:6 | | 1:6 | |
| reactant | ratio of | | | | | | | | |
| | MTHPA | | | | | | | | |
| Process | Injection of | Yes | | Yes | | No | | No | |
| parameter | nitrogen gas | | | | | | | | |
| | Temperature of | 160° | C. | 140° | C. | 140° | C. | 140° | C. |
| | isomerization | | | | | | | | |
| | reaction | | | | | | | | |
| | Relative amount | 1.5/100 | | 1.5/100 | | 1.5/100 | | 1.0/100 | |
| | of the alkaline | | | | | | | | |
| | catalyst | | | | | | | | |
| | Duration of | 3.5 | hours | 24 | hours | 24 | hours | 26 | hours |
| | isomerization | | | | | | | | |
| | reaction | | | | | | | | |
| Isomerization | Trans/cis | 4.22:1 | | 4.21:1 | | 4.24:1 | | 4.19:1 | |
| product | ratio of | | | | | | | | |
| | MTHPA | | | | | | | | |
| | Amount of | 1.55 | wt % | 1.52 | wt % | 4 | wt % | 3.72 | wt % |
| | methyltetrahydrophthalic | | | | | | | | |
| | anhydride dimer | | | | | | | | |

According to results from the experiments, the method for isomerizing methyltetrahydrophthalic anhydride of the present disclosure can prevent the formation of the methyltetrahydrophthalic anhydride dimer. Based on the total weight of the isomerization product being 100 wt %, the amount of the methyltetrahydrophthalic anhydride dimer can be lower than 3.5 wt %, preferably lower than 1.6 wt %.

According to Comparative Examples 1 and 2, on the condition of not injecting nitrogen gas, the higher an amount of the alkaline catalyst is, the shorter the duration of time required for the isomerization reaction is. However, oxygen gas and water vapor induce the dimerization of methyltetrahydrophthalic anhydride. Therefore, when a reaction rate of the isomerization reaction is high, a probability of the formation of methyltetrahydrophthalic anhydride dimer correspondingly increases.

In other words, when the isomerization reaction is carried out under a condition that nitrogen gas is not injected, the duration required for the process of the isomerization reaction is negatively proportional to the amount of the generated methyltetrahydrophthalic anhydride dimer.

Injection of nitrogen gas during the process of the isomerization reaction can overcome the problems of the dimerization of methyltetrahydrophthalic anhydride that occurs in Comparative Examples 1 and 2. According to Examples 1, 4, and 5, most oxygen gas and water vapor present in the liquid reactant are removed, such that double bonds of methyltetrahydrophthalic anhydride can be prevented from opening to form the methyltetrahydrophthalic anhydride dimer during the process of the isomerization reaction. Therefore, the larger the amount of the alkaline catalyst is, the shorter the duration for the process of the isomerization reaction is. Moreover, the amount of the generated methyltetrahydrophthalic anhydride dimer can be decreased.

In other words, on the condition of injecting nitrogen gas, the duration for the process of the isomerization reaction is positively proportional to the amount of the generated methyltetrahydrophthalic anhydride dimer that is generated. Accordingly, the method for isomerizing methyltetrahydrophthalic anhydride of the present disclosure can simultaneously achieve the effects of low time consumption and low impurity. In addition, the relationship that the duration for the process of the isomerization reaction is negatively proportional to the amount of the generated methyltetrahydrophthalic anhydride dimer is changed.

According to Examples 1, 2, and 6, the temperature for the isomerization reaction influences the duration required for the process of the isomerization reaction and the amount of the generated methyltetrahydrophthalic anhydride dimer. The higher the temperature for the isomerization reaction is, the shorter the duration for the process of the isomerization reaction is. However, the probability of the formation of the methyltetrahydrophthalic anhydride dimer increases under a higher temperature environment.

Therefore, in order to simultaneously achieve the effects of low time consumption and low impurity, the isomerization reaction is preferably processed at a temperature ranging from 145° C. to 155° C. Accordingly, the amount of the methyltetrahydrophthalic anhydride dimer can be lower than 1 wt %, preferably lower than 0.8 wt %, and more preferably lower than 0.6 wt %.

According to Examples 1 and 7, different manners to injecting nitrogen gas lead to different effects of removing oxygen gas and water vapor. Injecting nitrogen gas into the liquid reactant can directly remove oxygen gas and water vapor present in the liquid reactant. Compared to injecting nitrogen gas through a top of the reactor, directly injecting nitrogen gas into the liquid reactant has a better efficiency to remove oxygen gas and water vapor present in the system, so as to reduce the probability of the formation of the methyltetrahydrophthalic anhydride dimer.

### Beneficial Effects of the Embodiments

In conclusion, in the method for isomerizing methyltetrahydrophthalic anhydride of the present disclosure provided by the present disclosure, by virtue of “under a condition that nitrogen gas is injected, adding an alkaline catalyst into the liquid reactant for an isomerization reaction, so as to obtain an isomerization product,” the probability of the formation of the methyltetrahydrophthalic anhydride dimer can be reduced, thereby further enhancing the isomerization yield of methyltetrahydrophthalic anhydride.

Further, injecting nitrogen gas before the heating for the isomerization reaction can remove most oxygen gas and water vapor. Therefore, when the isomerization reaction is carried out, methyltetrahydrophthalic anhydride is prevented from opening double bonds to form the methyltetrahydrophthalic anhydride dimer. Since it is impossible to remove all oxygen gas and water vapor in the system, nitrogen gas is continuously injected throughout the overall process of the isomerization reaction so as to provide a good environment for the isomerization reaction.

Manners of injecting nitrogen gas can be directly injecting nitrogen gas into the liquid reactant in a form of bubbles to remove oxygen gas and water vapor, or indirectly injecting nitrogen gas through a top of the reactor to remove oxygen gas and water vapor accompanied by a flow of the nitrogen gas. According to the results of the present disclosure, both of the two manners can achieve the effect of removing oxygen gas and water vapor.

In the present disclosure, the injection of nitrogen gas changes the relationship of the duration required for the process of the isomerization reaction being negatively proportional to the amount of the generated methyltetrahydrophthalic anhydride dimer. Instead, the duration required for the process of the isomerization reaction is positively proportional to the amount of the generated methyltetrahydrophthalic anhydride dimer in the method for isomerizing methyltetrahydrophthalic anhydride of the present disclosure. Therefore, the method for isomerizing methyltetrahydrophthalic anhydride of the present disclosure has the effects of short time consumption and low impurity.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A method for isomerizing methyltetrahydrophthalic anhydride, comprising:
providing a liquid reactant which includes 3-methyltetrahydrophthalic anhydride and 4-methyltetrahydrophthalic anhydride; and
under a condition that nitrogen gas is injected, adding an alkaline catalyst into the liquid reactant for an isomerization reaction, so as to obtain an isomerization product;
wherein, based on a total weight of the isomerization product being 100 wt %, an amount of a methyltetrahydrophthalic anhydride dimer is lower than 1.6 wt %.

2. The method according to claim 1, wherein the liquid reactant is carried out for the isomerization reaction at a temperature ranging from 140° C. to 160° C.

3. The method according to claim 1, wherein the liquid reactant is carried out for the isomerization reaction under a condition that nitrogen gas is continuingly injected, and wherein a volume flow rate of the nitrogen gas ranges from 0.4 L/hour to 0.8 L/hour.

4. The method according to claim 1, wherein the nitrogen gas is injected into the liquid reactant before adding the alkaline catalyst into the liquid reactant for the isomerization reaction so as to remove oxygen gas and water vapor present in the liquid reactant.

5. The method according to claim 4, wherein the nitrogen gas is injected into the liquid reactant in a form of bubbles so as to remove oxygen gas and water vapor present in the liquid reactant.

6. The method according to claim 1, wherein the liquid reactant is carried out for the isomerization reaction in a reactor, and the nitrogen gas is injected into the reactor so as to remove oxygen gas and water vapor present in the reactor.

7. The method according to claim 1, wherein a weight ratio of trans-3-methyltetrahydrophthalic anhydride to cis-3-methyltetrahydrophthalic anhydride in the isomerization product ranges from 4:1 to 5:1.

8. The method according to claim 1, wherein, based on a total weight of the liquid reactant being 100 phr, an amount of the alkaline catalyst ranges from 0.5 phr to 1.5 phr.

9. The method according to claim 1, wherein a weight ratio of trans-3-methyltetrahydrophthalic anhydride to cis-3-methyltetrahydrophthalic anhydride in the liquid reactant ranges from 1:4 to 1:7.

10. The method according to claim 1, wherein a weight ratio of 3-methyltetrahydrophthalic anhydride to 4-methyltetrahydrophthalic anhydride in the liquid reactant ranges from 7:3 to 3:7.
